(19) United States
(12) Patent Application Publication
(10) Pub. No.: US 2026/0198402 A1
(43) Pub. Date: Jul. 16, 2026

(54) IN SITU DETERMINING OF SOIL PROPERTIES AND CONTROL OF WORK MACHINE OPERATIONS USING THE SAME

(71) Applicant: Deere & Company, Moline, IL (US)

(72) Inventors: Lance S. Conway, New Haven, MO (US); Timothy A. Wilcox, Cissna Park, IL (US); Broughton C. Boydell, Biniguy NSW (AU); Bradley W. Van De Woestyne, West Des Moines, IA (US); Peter Kyveryga, Urbandale, IA (US)

(21) Appl. No.: 19/022,005

(22) Filed: Jan. 15, 2025

(51) Int. Cl. A01B 79/00; G01N 21/31; G01N 27/04; G01N 33/24

(52) U.S. Cl. CPC A01B 79/005; G01N 21/31; G01N 27/043; G01N 33/245; G01N 33/246

(57) ABSTRACT

Disclosed herein are multiple sensor instruments for simultaneously or near simultaneously measuring two or more soil properties in situ. Also disclosed herein are controllers capable of communicating with the multiple sensor instruments and capable of determining one or more indirectly assessed soil properties based on the properties directly measured by the sensors. Also disclosed herein are instrumented systems combining the sensors and controller with one or more work tools which may be operationally controlled by the controller based on the directly measured or indirectly assessed soil properties, and methods for controlling a work operation using the same. Such systems may be further configured to communicate with an external data source, containing data that may be used along with the directly measured or indirectly assessed properties in controlling the work operation.

A portion of the disclosure of this patent document contains material that is subject to copyright protection. The copyright owner has no objection to the reproduction of the patent document or the patent disclosure, as it appears in the U.S. Patent and Trademark Office patent file or records, but otherwise reserves all copyright rights whatsoever.

### BACKGROUND

The present disclosure relates generally to instrument systems for determining soil properties based on in situ measurements and control methods using such systems. The present disclosure more particularly relates to systems for controlling work machine operations, for example relating to seeding, tillage, spraying, and the like, including control of advance speed, steering, and/or actuation of work tools for treatment of a ground surface, introducing a granular material (for example, seeds, solid fertilizer, etc.) to the soil of a worksite, or the like.

Typically, operations at a work site such as tilling, planting, and fertilization, involve the measurement of one or more soil properties that inform or shape operational parameters of the operation, such as planting depth, planting rate, pesticide rate, fertilization rate, tilling depth, and tilling angle. However, the most relevant properties often cannot be measured directly, and must instead be calculated or estimated based on one or more properties that are amenable to direct in situ measurement.

These direct measurements may, however, be subject to sources of error that reduce the accuracy of subsequent calculations or estimates. The scale of this error may be reduced, or in some cases, even eliminated, by directly measuring additional in situ properties that allow for appropriate correction of error based on the measurement of such additional properties.

What is needed, then, is an instrument capable of simultaneously measuring two, three, or more in situ properties, and control mechanisms for calculating or estimating operationally relevant soil characteristics based on the in situ multiple property measurements.

### BRIEF SUMMARY

Disclosed herein are examples of multiple sensor instruments. These multiple sensor instruments may include two or more different kinds of sensors for the measurement of in situ soil properties. The sensors may be in communication with a control unit or processor that fuses the respective sensor outputs and calculates, estimates, or derives one or more soil properties based on the directly measured values. In some examples, the calculated, estimated, or derived values may be further used to calculate or predict one or more operationally significant values. The controller may also be in communication with one or more work tools and configured to control the work tools based on the directly measured properties, the calculated, estimated, or derived properties, the calculated or predicted operationally significant values, or a combination thereof. Work tools within the scope of the present disclosure may include, without limitation and unless otherwise specifically noted herein, ground-engaging implements such as for example furrow openers, crop-engaging implements such as with combine harvesters, sprayers for treatment of a work area, and the like as may be appreciated by one of skill in the art.

In a first exemplary embodiment, a method as disclosed herein method for controlling one or more operations of a work machine in a work site comprises: generating a first sensor signal representative of a first soil characteristic at a work location and a second sensor signal representative of a second soil characteristic at the work location; calculating a soil moisture content from first soil characteristic and the second soil characteristic, and calculating a soil texture (e.g., sand/silt/clay) value from the first soil characteristic and the second soil characteristic; estimating one or more additional soil characteristics based at least on the soil moisture content and the soil texture value; generating respective values for one or more operational settings, based on at least one of the first soil characteristic, the second soil characteristic, and the one or more further soil characteristics; and controlling at least one operation of the work machine based on the generated one or more operational settings relative to current actual values for the one or more operational settings.

In one exemplary aspect according to the above-referenced first method embodiment, the second soil characteristic may be electrical conductivity.

In another exemplary aspect according to the above-referenced first method embodiment, a third soil characteristic may be determined based on measurements from a third sensor, and the one or more further soil characteristics may be estimated further based at least on the third soil characteristic.

In another exemplary aspect according to the above-referenced first method embodiment, the third soil characteristic may be a measured absorption value for the absorption of light with wavelengths ranging from UV-VIS-NIR-MIR (~200-4000 nm), and wherein the calculation of at least one of the soil moisture content and the soil texture value is further based on the measured absorption value.

In another exemplary aspect according to the above-referenced first method embodiment, publicly available soil and/or terrain geographic data and/or previously collected proximally-sensed soil sensor information associated with the work site may be obtained, and the one or more further soil characteristics may be estimated further based on the obtained soil and/or terrain geographic data and/or previously collected proximally-sensed soil sensor information for respective locations in the work site. An example of publicly available soil geographic data may include data selectively retrieved from a SSURGO database.

In another exemplary aspect according to the above-referenced first method embodiment, the first soil characteristic may be a dielectric constant, and the measuring of the dielectric constant and the measuring of electrical conductivity may be conducted at a plurality of locations and a soil moisture content, and a soil texture value may be calculated for each location of the plurality of locations.

In another exemplary aspect according to the above-referenced first method embodiment, each of the steps in the method may be performed by the work machine in association with a current operation. In other words, the measuring of the soil characteristics and control steps may be performed substantially in real time during traverse of the work site by the work machine.

In another exemplary aspect according to the above-referenced first method embodiment, a target planting depth map for a work site may be prepared including a plurality of locations during a first process associated with the work site, based on a plurality of modeled seed planting depths corresponding to the plurality of locations, and at least one operation of the work machine is controlled during a second process associated with the work site and based on the target planting depth map. In other words, various steps including the measuring of the soil characteristics may be performed by a first work machine and then other steps including the control steps may be performed by a second work machine at a later time using the prepared map. Alternatively, the various steps may be performed by the same work machine but at different times, again within the scope of the present disclosure.

In another exemplary aspect according to the above-referenced first method embodiment, a control signal may be transmitted at each location of the plurality of locations to control the operation of a seeding implement to plant a seed at the modeled seed planting depth corresponding to that location.

In another exemplary aspect according to the above-referenced first method embodiment, the planting depth map and corresponding control signals may be stored, and the planting depth map and corresponding control signals associated with the working area.

In another exemplary aspect according to the above-referenced first method embodiment, one or more additional sensors may be utilized to measure one or more additional soil characteristics and generate one or more corresponding additional signals representative of the one or more additional soil characteristics.

In another exemplary aspect according to the above-referenced first method embodiment, the step of calculating the soil moisture content from the first soil characteristic and the second soil characteristic, and calculating the soil texture value from the first soil characteristic and the second soil characteristic may further include modifying at least one of the soil moisture content and the soil texture value based on the one or more additional signals.

In another exemplary aspect according to the above-referenced first method embodiment, the one or more further soil characteristics may include at least one of plant-available water level or soil water tension value.

In another exemplary aspect according to the above-referenced first method embodiment, the step of predicting the plant-available water level or the soil water tension value may further comprise comparing the soil moisture content or the soil texture value against a corpus of previously measured soil moisture contents or soil texture values correlated to known plant-available water levels or soil water tension values.

In another exemplary aspect according to the above-referenced first method embodiment, the soil moisture content or the soil texture value may be added to a corpus of previously measured soil moisture contents or soil texture values and the soil moisture content or the soil texture value correlated to a modeled plant-available water level or soil water tension value.

In another exemplary aspect according to the above-referenced first method embodiment, the work machine may comprise a tillage implement, wherein the operational setting includes a depth or an angle at which a tiller engages the soil based at least in part on the soil moisture content, the soil texture value, or the one or more further soil characteristics.

In another exemplary aspect according to the above-referenced first method embodiment, the work machine may comprise a product applicator, and a pesticide rate may be calculated based on at least one of the soil texture value and a calculated organic matter content value, and a pesticide treatment applied in an amount based on the calculated pesticide rate.

In another exemplary aspect according to the above-referenced first method embodiment, a predicted emergence behavior may be modeled for a seed planted in a soil having the predicted plant-available water level and soil water tension value.

In another exemplary aspect according to the above-referenced first method embodiment, the work machine may comprise a fertilizer applicator, wherein the method may further comprise calculating a starter fertilizer rate based on at least one of the plant-available water level or the soil water tension value and applying a fertilizer to the soil in an amount based on the starter fertilizer rate.

In another embodiment as disclosed herein, a device may be configured to be attached to a work machine, the device comprising: a first sensor configured to generate output signals representative of a dielectric constant of soil; a second sensor configured to generate output signals representative of soil conductivity; and a controller operably connected to the first and second sensors, and a seed applicator or a tiller, and configured to direct performance of steps according to the above-referenced first method embodiment and optionally one or more of the aspects thereof.

In another embodiment as disclosed herein, a system may be provided for controlling one or more operations of a work machine in a work site, the system comprising: a first sensor configured to generate output signals representative of a dielectric constant of soil; a second sensor configured to generate output signals representative of soil conductivity; and one or more processors operably connected to the first and second sensors, and configured to direct performance of steps according to the above-referenced first method embodiment and optionally one or more of the aspects thereof, wherein the dielectric constant is the first soil characteristic and the soil conductivity is the second soil characteristic.

In another embodiment, a method is disclosed herein for controlling an instrumented seed applicator having a capacitance sensor and a conductivity sensor, and comprises: transmitting a control signal to engage a seed applicator with a soil at a first depth at a worksite location; measuring a capacitance value and a conductivity value of the soil at the first depth; calculating a soil moisture content from the capacitance value and the conductivity value and calculating a soil texture value from the capacitance value and the conductivity value; predicting a plant-available water level and a soil water tension value from the soil moisture content and the soil texture value; comparing at least one of the plant-available water level or the soil water tension value to a target value for the plant-available water level or the soil water tension value; and controlling the depth of the seed applicator within the soil in response to the comparing of the at least one of the plant-available water level or the soil water tension value to a target value for the plant-available water level or the soil water tension value.

In one exemplary aspect according to the above-referenced second method embodiment, a seed may be introduced to the soil, when the plant-available water level matches or exceeds the target value of plant-available water level, or when the soil water tension value matches or exceeds the target value of the soil water tension value.

In another exemplary aspect according to the above-referenced second method embodiment, the seed applicator may be advanced to a second depth deeper than the first depth when the plant-available water level is less than the target value of plant-available water level, or when the soil water tension value is less than the target value of the soil water tension value, and recalculating the soil moisture content from the capacitance value and the conductivity value and recalculating calculating the soil texture value from the capacitance value and the conductivity value.

In another exemplary aspect according to the above-referenced second method embodiment, the worksite location may be a first worksite location, wherein the method may further comprise advancing the seed applicator from the first worksite location to a second worksite location and at the second worksite location: measuring a capacitance value and a conductivity value of the soil at the first depth; calculating a soil moisture content from the capacitance value and the conductivity value and calculating a soil texture value from the capacitance value and the conductivity value; predicting a plant-available water level and a soil water tension value from the soil moisture content and the soil texture value; comparing at least one of the plant-available water level or the soil water tension value to a target value for the plant-available water level or the soil water tension value; and controlling the depth of the seed applicator within the soil in response to the comparing of the at least one of the plant-available water level or the soil water tension value to a target value for the plant-available water level or the soil water tension value.

Numerous objects, features and advantages of the embodiments set forth herein will be readily apparent to those skilled in the art upon reading of the following disclosure when taken in conjunction with the accompanying drawings.

### BRIEF DESCRIPTION OF THE DRAWINGS

FIG. 1 is a top view schematic of a work vehicle and a work tool according to one aspect of the present disclosure.

FIG. 2A is a schematic representation of a multiple sensor instrument according to one aspect of the present disclosure.

FIG. 2B is a schematic representation of a multiple sensor instrument according to another aspect of the present disclosure.

FIG. 2C is a schematic representation of a multiple sensor instrument according to another aspect of the present disclosure.

FIG. 3 is a graph showing the very near infrared absorption value of representative soil samples according to one aspect of the present disclosure.

FIG. 4 is a schematic of an instrument controlled system according to one aspect of the present disclosure.

FIG. 5 is a flowchart depicting an example work operation utilizing an instrument controlled system according to one aspect of the present disclosure.

### DETAILED DESCRIPTION OF THE INVENTION

The following explanations of terms are provided to better describe the present disclosure and to guide those of ordinary skill in the art in the practice of the present disclosure. As used herein, “comprising” means “including” and the singular forms “a” or “an” or “the” include plural references unless the context clearly dictates otherwise. The term “or” refers to a single element of stated alternative elements or a combination of two or more elements unless the context clearly indicates otherwise.

Unless explained otherwise, all technical and scientific terms used herein have the same meaning as commonly understood to one of ordinary skill in the art to which this disclosure belongs. Although methods and compounds similar or equivalent to those described herein can be used in the practice or testing of the present disclosure, suitable methods and compounds are described below. The compounds, methods, and examples are illustrative only and not intended to be limiting, unless otherwise indicated. Other features of the disclosure are apparent from the following detailed description and the claims.

Unless otherwise indicated, all numbers expressing quantities of components, percentages, temperatures, times, and so forth, as used in the specification or claims are to be understood as being modified by the term “about.” Accordingly, unless otherwise indicated, implicitly or explicitly, the numerical parameters set forth are approximations that can depend on the desired properties sought and/or limits of detection under standard test conditions/methods. When directly and explicitly distinguishing embodiments from discussed prior art, the embodiment numbers are not approximate unless the word “about” is recited. Furthermore, not all alternatives recited herein are equivalents.

The desired operational parameters for a number of operations at a worksite may depend on a number of soil attributes. For example, seed planting depth may be governed by the amount of water that is available to a seed at a given depth in a given location at the worksite and may also be governed by the water tension in the soil at that location. However, such values are difficult to directly measure. Instead, it may be necessary to calculate, estimate, or predict these values based on one or more soil attributes that can be directly measured.

General, site-wide knowledge of these directly soil attributes may provide correspondingly general guidance on optimal planting depth across that worksite but may not account for local variations in the general site-wide conditions. It may therefore be useful to provide a location specific, site-associated map of soil attributes that provides guidance for individualized or localized planting conditions for individual planting locations at the worksite.

Even this location specific, site associated map of attributes may, however, be insufficient to account for conditions or soil attributes that may change over time. To address these characteristics, it may be necessary to measure the attributes of interest shortly prior to planting.

Both of these objectives can be accomplished by an in situ measurement of one or more relevant soil attributes during the planting operation. Such measurements may, for example, be accomplished with the use of one or more sensors brought into contact or engagement with the soil. In some examples, these sensors can be directly associated with a work machine, such as a work vehicle that is operational in a worksite, but it will be appreciated that the sensors may be operated in a standalone fashion.

In some cases, the type of sensor used to measure a relevant soil attribute may be subject to a source of error, which may limit the accuracy (and thus the analytical value) of any measurement of the soil attribute in question. However, in many cases, different kinds of sensors will be subject to different sources and types of error. In such cases, it may be advantageous and desirable to combine two or more different sensors, each configured to measure or otherwise enable estimation of a different soil attribute and each subject to a different source or type of measurement error. Thus, data from one sensor can correct for measurement errors in other sensors, and vice versa.

It will be appreciated that, while in situ measurement of one or more relevant soil attributes is presented herein primarily in association with a planting operation (that is, the in situ measurements are taken during or immediately before a planting operation), it is to be understood that the in-situ measurements can alternatively or additionally be taken independently of any planting operation. For example, such in-situ measurements can be taken to establish a location specific site associated map of relevant attributes. Such in situ measurements can also be taken to add to a corpus of other or similar soil attribute data, which may optionally be used to form one or more correlative and/or predictive learning models and/or algorithms related to the one or more soil attributes. Any such learning model and/or algorithm may be used alone or in combination with further in situ measurements to predict soil characteristics, particularly soil attributes associated with a worksite.

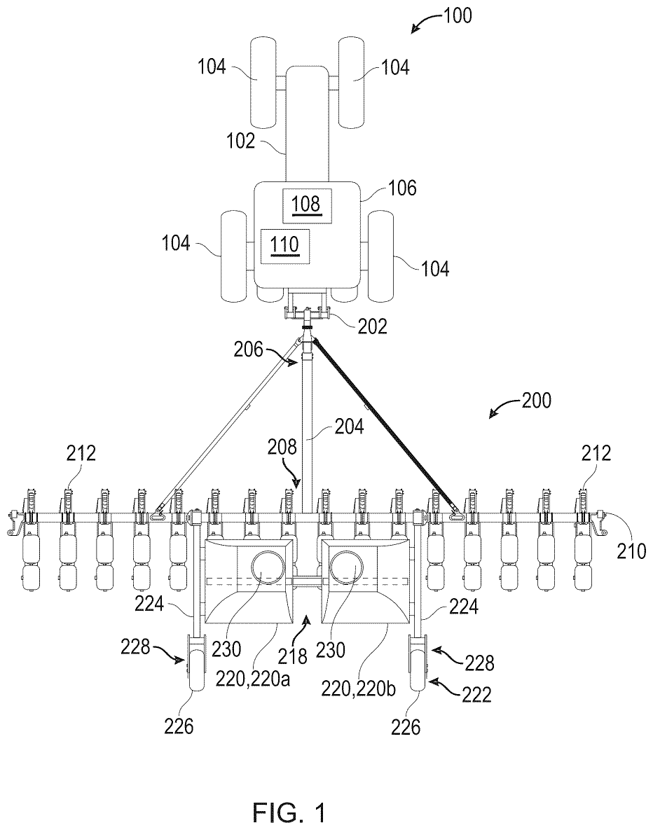

Referring now to the drawings, FIG. 1 shows a work vehicle 100, which according to various aspects of the present disclosure can be used with a work tool, such as a seed applicator, a fertilizer applicator, or a tilling instrument.

As shown in FIG. 1, the work vehicle 100 can include a vehicle chassis 102 and ground engagement features 104 attached to the vehicle chassis 102. As shown in FIG. 1, the ground engagement features 104 can be wheels in some examples, but it will be appreciated that in other examples, the ground engagement features 104 can have a different form, such as tracks or treads.

The work vehicle 100 also comprises an operator cab 106, mounted to the vehicle chassis 102. The operator cab 106 can include one or more features for the operation of the work vehicle 100, including steering and driving controls 108 and a control station 110, which according to a general example are positioned within reach of a user during the operation of the work vehicle 100.

In some examples, the work vehicle 100 is configured to attach to and assist in the operation of various working tools. In such examples, the work vehicle 100 can be attached or hitched to a work tool 200, such that the work vehicle 100 can move the work tool 200 within a worksite. According to some aspects of the present disclosure, the work tool 200 can be seeder (that is, a tool configured to introduce seeds to the soil of a work site), but in other aspects of the present disclosure, the work tool 200 can be or include some other sort of work tool, such as a fertilizer applicator, a row cleaner, a tiller, or a closing wheel.

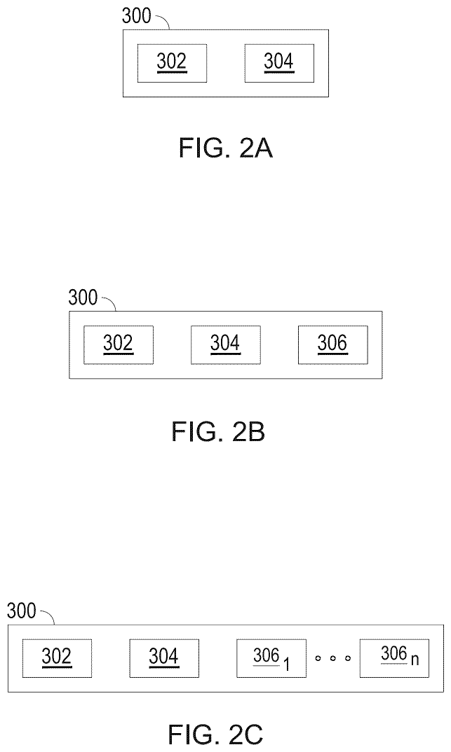

Turning now to FIG. 2, an example of multisensor instrument 300 is schematically shown. It will be appreciated that, while the multisensor instrument 300 will be discussed herein as associated with the work tool 200, the multisensor instrument 300 can be associated with either or both of the work vehicle 100 and the work tool 200.

According to one aspect of the present disclosure, the multisensor instrument 300 comprises a first sensor 302 and a second sensor 304. Both the first sensor 302 and the second sensor 304 can be a different kind of sensor, such that the multisensor instrument 300 includes two or more different kinds of sensors. While the disclosure herein will primarily concern a multisensor instrument 300 including an electrical capacitance sensor and an electrical conductivity sensor, it will be appreciated that other types of sensors, such as visible and near infrared (VNIR) sensors, temperature sensors, optical sensors, or any other sensors suitable for measuring one or more soil attributes that are relevant to determining the parameters of a planting operation.

According to one aspect of the present disclosure, the multisensor instrument 300 can include one or more additional sensors 306. For example, in some aspects of the present disclosure the multisensor instrument 300 can include an additional sensor 306 in addition to the first sensor 302 and the second sensor 304. In some aspects of the present disclosure, the multisensor instrument 300 can include further sensors, such that the multisensor instrument 300 includes n sensors: first sensor 302 through an n^{th} sensor 306n. These additional sensors 306 through 306n can be any type of sensor previously introduced or otherwise capable of generating output signals representative of soil characteristics, attributes, or the like as may be relevant to a system and method as disclosed herein. In various embodiments, one sensing technology may be utilized to measure, estimate, or predict one or more soil characteristics, attributes, or the like. In various embodiments, each soil characteristic, attribute, or the like may be measured, estimated, or predicted based on a respective sensor output, or one or more of the soil characteristics, attributes, or the like may be measured, estimated, or predicted based on redundant output signals from two or more sensing technologies.

Exemplary such technologies may include, without limitation, signals representative of dielectric constant, electrical conductivity, visible and near infrared reflectance, mid-infrared reflectance, apparent electrical conductivity and/or impedance, light absorption, capacitance, ground penetrating radar (GPR), gamma-ray spectroscopy, laser induced breakdown spectroscopy (LIBS), x-ray fluorescence spectroscopy (XRF), wet chemistry analysis, visible and near infrared (VNIR) sensors, temperature sensors, optical sensors such as in-furrow cameras, etc.

According to one aspect of the present disclosure each of the sensors (for example, the first sensor 302, the second sensor 304, the additional sensor 306, and each further sensor to the nth sensor 306n) of the multisensor instrument 300 can be designed to directly measure a different primary soil characteristic. For example, if the first sensor 302 is a capacitance sensor, first sensor 302 can directly measure an electrical capacitance value of the soil, and if the second sensor 304 is a conductivity sensor, the second sensor 304 can directly measure a conductivity value of the soil. It will be appreciated that other types of sensors previously discussed may each directly measure a different primary soil characteristic corresponding to different kinds of sensors.

The primary soil characteristics of the soil can, according to some aspects of the present disclosure, be related to calculated soil properties. Calculated soil properties, such as moisture content, texture, organic matter content, or salinity are properties of the soil that often cannot be directly measured, but which can be functionally important for establishing one or more operational parameters of a work procedure, such as a seeding operation or product application operation. As such, the various primary soil characteristics measured by the multisensor instrument 300 can thereafter be used to calculate one or more corresponding calculated soil properties.

According to one aspect of the present disclosure, the relevant calculated soil properties may include soil moisture content and soil texture. Generally, soil moisture content is a calculated soil property that has a great effect on the dielectric properties of the soil, and therefore dielectric constant measurements may be used to subsequently calculate or determine the soil moisture content. Likewise, soil texture is a calculated soil property with a great effect on the electrical conductivity of the soil, and therefore electrical conductivity measurements may be used to subsequently calculate or determine the texture of the soil.

However, other factors can introduce errors in the calculation and/or determination of the calculated soil properties to be calculated. For example, the dielectric constant of soil may be primarily affected by the moisture content of the soil, but may also be affected by the texture, soil bulk density, salinity, and/or temperature of the soil. Thus, these additional properties such as texture, soil bulk density, salinity, and/or temperature of the soil may introduce error into the calculation and/or determination of the moisture content of the soil. Likewise, the conductivity of a soil may be primarily affected by the soil texture, but may also be affected by the moisture content, salinity, or density of the soil. Thus, these additional properties such as moisture content, salinity, and/or density of the soil can introduce errors into the calculation and/or determination of the texture of the soil.

In such examples, it may be possible to correct for the error introduced to the calculation and/or determination of a calculated soil property through one or more of these additional properties if the additional property is directly measured, and an appropriate corrective factor determined based on the measurement of these one or more additional properties can be calculated and incorporated into the calculation and/or determination of the calculated soil property in question. Thus, it may be advantageous to use a multisensor instrument 300, wherein the various sensors (for example, the first sensor 302, the second sensor 304, and the additional sensors 306) directly measure primary soil characteristics that can be used to correct for any error in the calculation of calculated soil properties based on the directly measured primary soil characteristics of one or more different sensors.

For example, according to one aspect of the present disclosure, the multisensor instrument 300 can include a first sensor 302 that is a dielectric constant sensor and a second sensor 304 that is an electrical conductivity sensor. When the multisensor instrument 300 is engaged with the soil, the first sensor 302 can measure the capacitance/dielectric constant of the soil and the second sensor 304 can measure the electrical conductivity of the soil. Thereafter, when the moisture content of the soil is calculated based on the directly measured capacitance/dielectric constant of the soil, the electrical conductivity of the soil will also be known and can be used to estimate soil texture. With an estimate for soil texture available, any error in the moisture content of the soil resulting from the soil texture can be at least partially mitigated with a correction factor based on the estimated soil texture. Likewise, when the texture of the soil is calculated based on the electrical conductivity of the soil, availability of conductivity data will allow an estimate of the volumetric water content of the soil. In turn, this can allow the calculation of the texture of the soil to incorporate a correction factor for the estimated volumetric water content.

It will be appreciated that according to some aspects of the present invention, data may be continuously collected by the first sensor 302 and the second sensor 304 (that is, the capacitance sensor and the conductivity sensor in the example discussed), and the collected data from each sensor can be combined to simultaneously update calculations for the volumetric water content and the soil texture at the measurement site. Thus, the estimated volumetric water content will be calculated based on both the capacitance data and the conductivity data, and the estimated soil texture will be calculated based on both the conductivity data and the capacitance data.

According to some aspects of the present disclosure, one or more additional sensors 306 may also be included in the multisensor instrument 300 and used to directly measure one or more additional soil characteristics (sometimes called to further soil characteristics) that may further correct for one or more corresponding sources of error. For example, in some aspects of the present disclosure, the multisensor instrument 300 may include a one or more additional sensors 306 that is a VNIR absorption sensor. The VNIR absorption sensor can, in such examples, provide absorption data (that is, a measured absorption value) for the soil sample, which may be combined with data from the other sensors (for example, the first sensor 302 and the second sensor 304) of the multisensor instrument 300 to further correct for error measurements. Advantageously, the VNIR absorption of a soil sample is primarily affected my moisture content but can be affected as well by organic material content and soil texture, and as such VNIR data may be used to provide adjustment factors for the measurement of both soil texture and moisture content, in addition to providing information about the organic material content of the soil.

According to one aspect of the present disclosure, the VNIR absorption sensor of the multisensor instrument 300 can produce VNIR absorption data that is combined with capacitance data from the capacitance sensor, and conductivity data from the electrical conductivity sensor, and the calculated soil properties (that is, the volumetric water content and the soil texture) can be calculated from the combined capacitance data, conductivity data, and VNIR absorption data. It will also be readily appreciated that, in such examples, an estimate of the organic material content of the soil may also be provided.

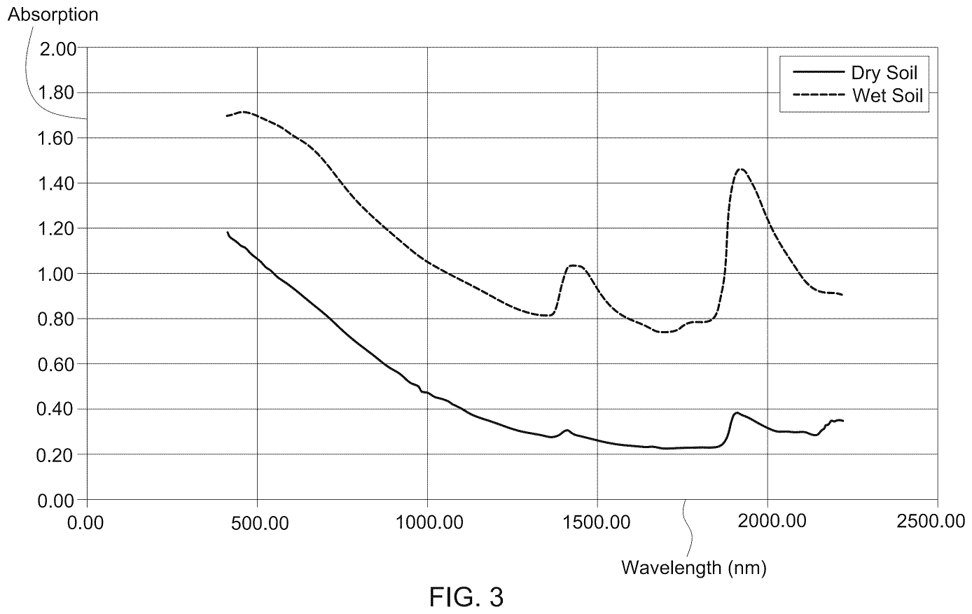

According to one aspect of the present disclosure, the VNIR sensor can be configured to gather absorption data across a particular range of wavelengths of light, with absorption values being dependent in part on soil conditions or properties (e.g., wet vs. dry). For example, as illustrated in FIG. 3, the VNIR sensor may be configured to measure VNIR absorption at wavelengths of 450 nm to about 2500 nm, or more particularly, 450 nm to 2200 nm.

While detailed reference has been made herein to specific calculated soil properties, such as the volumetric water content, the texture, and the organic material content of the soil, it will be readily appreciated that other properties that may be directly measured (such as temperature), and other calculated soil properties may be determined by including additional diverse sensors in the multisensor instrument 300.

According to some aspects of the present disclosure, the calculated soil properties may then be used to establish one or more operationally significant parameters. For example, plant-available volumetric water level of a soil is a factor of both the volumetric water content and the soil texture. With both volumetric water content and soil texture values established to a high degree of accuracy by the various sensors of the multisensor instrument 300, a specific determination of how much water at a given site will be available to plants may be made. Likewise, the availability of volumetric water content and soil texture values may allow the soil water tension at a given site to be accurately determined. Advantageously, the ability to determine (for example by calculation or modeling) operationally significant parameters such as plant-available water level and soil water tension can allow for various operations to be controlled to a greater degree of utility and precision by systems incorporating the multisensor instrument 300, as disclosed herein.

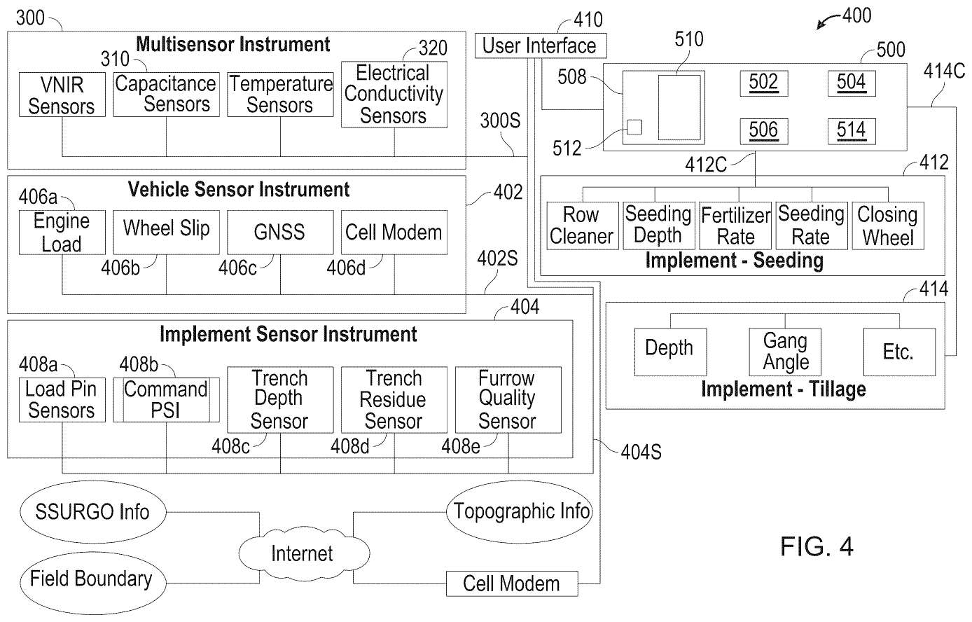

Some aspects of the present disclosure concern instrumented systems including a multisensor instrument, such as the multisensor instrument 300. As shown in FIG. 4, an instrumented system 400 can include the multisensor instrument 300, as well as one or more additional sensor instruments. For example, the instrumented system 400 can also include a vehicle sensor instrument 402 and an implement sensor instrument 404.

As shown in FIG. 4, the vehicle sensor instrument 402 can include vehicle sensors 406 that provide data on the operational state or of various systems of the working vehicle (such as the work vehicle 100) or the position of the working vehicle. Such data can include data on, for example, engine load, wheel slip, global navigation satellite system (GNSS) position, cell modem data, and data on any other parameter useful for identifying the condition, state, or position of the working vehicle. Each of these parameters may be associated with a specific sensor that measures the parameter. For example, as illustrated in FIG. 4, the vehicle sensor instrument 402 can include an engine load sensor 406a, a wheel slip sensor 406b, a GNSS position sensor 406c, and a cell modem sensor 406d, which collect data on engine load, wheel slip, GNSS position, and cell network information, respectively.

With continued reference to FIG. 4, the implement sensor instrument 404 can include one or more implement sensors 408. The one or more implement sensors 408 can measure and collect data on one or more corresponding implements associated with a vehicle such as the work vehicle 100, or a work tool (such as the work tool 200) attached to the work vehicle. These implement sensors 408 can collect data on one or more parameters affecting one or more implements of the work vehicle 100, or a work tool attached to the work vehicle. For example, as illustrated in FIG. 4, the implement sensor instrument 404 can include a load pin sensor 408a, a command PSI sensor 408b, a trench depth sensor 408c, a trench residue sensor 408d, a furrow quality sensor 408e, or any combination thereof. Additionally, the implement sensor instrument 404 can include any other sensors suitable for measuring or collecting data on one or more additional corresponding implements that may be associated with the work vehicle or work tool.

The multisensor instrument 300, the vehicle sensor instrument 402, and the implement sensor instrument 404 of the instrumented system 400 can each be in communication with a controller, such as the controller 500 shown in FIG. 4.

The controller 500 includes or may be associated with a processor 502, a computer readable medium 504, a database 506 and an input/output module or control panel 308 having a display 510. The control panel 508 may be a part of the control station 110 in the operator cab 106, or part of a user interface unique to the 400, as discussed in greater detail herein, or can be separate from either the control station 110 or the user interface. An input/output device 512, such as a keyboard, joystick, touchscreen, mobile device, or other user interface, can be provided so that a human operator may input instructions to the controller 500. It is understood that the controller 300 described herein may be a single controller having the described functionality, or it may include multiple controllers wherein the described functionality is distributed among the multiple controllers. Some or all of the controllers may be located at a location other than the work vehicle and be connected wirelessly.

Various operations, steps or algorithms as described in connection with the controller 500 can be embodied directly in hardware, in a computer program product 514 such as a software module executed by the processor 502, or in a combination of the two. The computer program product 514 can reside in RAM memory, flash memory, ROM memory, EPROM memory, EEPROM memory, registers, hard disk, a removable disk, or any other form of computer-readable medium 504 known in the art. An exemplary computer-readable medium 504 can be coupled to the processor 502 such that the processor 502 can read information from, and write information to, the memory/storage medium. In the alternative, the medium can be integral to the processor. The processor and the medium can reside in an application specific integrated circuit (ASIC). The ASIC can reside in a user terminal. In the alternative, the processor and the medium can reside as discrete components in a user terminal.

The term “processor” as used herein may refer to at least general-purpose or specific-purpose processing devices and/or logic as may be understood by one of skill in the art, including but not limited to a microprocessor, a microcontroller, a state machine, and the like. A processor can also be implemented as a combination of computing devices, e.g., a combination of a DSP and a microprocessor, a plurality of microprocessors, one or more microprocessors in conjunction with a DSP core, or any other such configuration.

According to one aspect of the present disclosure, illustrated in FIG. 4, the controller 500 is configured to receive one or more input signals and to produce and transmit one or more output signals in response to or based in part on the input signals. For example, soil sensor signals 300S from the sensors of the multisensor instrument 300 can be sent to the controller 500. Similarly, vehicle sensor signals 402S and 404S can be transmitted to the controller 500 from the vehicle sensor instrument 402 and the implement sensor instrument 404, respectively. It will be appreciated that, while the signals 300S, 402S, and 404S have been depicted in FIG. 4 as signals that combine data from each of the respective instruments connected thereto, each of the sensors may similarly transmit an individual signal to the controller. For example, if the multisensor instrument contains a capacitance sensor and a conductivity sensor, it may send a first sensor signal representative of the capacitance sensor 310 and a second sensor signal representative of the conductivity sensor 320.

According to one aspect of the present disclosure, the instrumented system 400 can also include a user interface 410, as illustrated in FIG. 4. The multisensor instrument 300, the vehicle sensor instrument 402, the 404, and the controller 500 can be in communication with the user interface 410. The user interface 410 can be configured to present data from any combination of sensors of one or more of the multisensor instrument 300, the vehicle sensor instrument 402, or the implement sensor instrument 404. The user interface 410 can also include or incorporate an input/output module or control panel, such as the control panel 308 previously disclosed. The input/output module or control panel can be configured to receive user input, which can thereafter be transmitted to one or more of the other components of the instrumented system 400, such as the controller 500.

The controller 500 can also be in communication with one or more work tools, and configured to provide control signals that control the operation of the one or more work tools. Such work tools can include seeding implements, tilling implements, or any other implements suitable for control by means of one or more signals transmitted from the controller 500. For example, as illustrated in FIG. 4, according to one aspect of the present disclosure, the instrumented system 400 can include a seeding tool 412.

The seeding tool 412 can include one or more tools, including a row cleaner, a seed applicator, a fertilizer applicator, and a closing wheel, each of which may be controlled according to one or more parameters of a seeding tool control signal 412C transmitted to the seeding tool 412 from the controller 500. In such examples, the controller can generate one or more operational settings for one or more of the tools associated with the seeding tool 412. The operational settings can be based on one or more directly measured soil properties, calculated soil properties, or operationally significant parameters, and can govern one or more associated implement control actions. For example, the controller 500 can calculate a target planting depth, a target fertilizer rate, a starter fertilizer rate, a target seeding rate, a target closing wheel force, a target gauge wheel load, or any combination thereof from the data available from instruments such as the multisensor instrument 300, the vehicle sensor instrument 402, or the implement sensor instrument 404. Thus, the seeding tool control signal 412C can include implement control actions governing starter fertilizer application, fertilizer application rates, seed application rates, seed application depth, and closing wheel force for the one or more tools associated with the seeding tool 412.

Likewise, as illustrated in FIG. 4, the instrumented system 400 can include a tilling instrument 414. The tilling instrument 414 can operate according to one or more operational parameters, including tillage depth and gang angle. These operational parameters can be set by implement control actions, determined by the controller 500 based on the data available from instruments such as the multisensor instrument 300, the vehicle sensor instrument 402, or the implement sensor instrument 404, and thereafter transmitted from the controller 500 to the tilling instrument 414 by a tilling instrument control signal 414C. Thus, the operational parameters of the tilling instrument 414, such as the tilling depth and the gang angle of the tilling instrument can be controlled based on one or more of the directly measured soil properties, calculated soil properties, or operationally significant parameters available to the controller 500 through the sensor instruments, including the multisensor instrument 300, the vehicle sensor instrument 402, and the implement sensor instrument 404.

With continued reference to FIG. 4, the instrumented system 400 can also be connected to the internet, for example, through a cellular connection. The internet connection can provide the user (for example, through the user interface 410) or the controller 500 access to one or more sets of data which may further be used to implement control actions of any implements operationally connected to the controller.

For example, the instrumented system 400 can, through the internet connection, have access to externally stored site specific data, including field boundaries, soil conditions, and topographic information. The instrumented system 400 can also have access to one or more databases of relevant data, such as the soil survey geographic database (SSURGO), or a database containing site specific data for a plurality of worksites.

In such examples, the access to externally stored site specific data may be used alone (for example, as part of a predictive model), or in combination with any of the data gathered by the instruments (that is, the multisensor instrument 300, the vehicle sensor instrument 402, or the implement sensor instrument 404), or with any calculations made based on such data, to further refine one or more operational parameters to control the operation of a working tool (such as a seeding implement or tilling implement). For example, if the externally stored data includes a site map with estimated or predicted values for target tilling gang angle, target tilling depth, target planting depth, pesticide application rate, target fertilizer amount, target row closure force, target seed application rate, or any combination thereof, associated with different work locations within the site map further in situ measurements, particularly by the multisensor instrument 300 taken at any work location at the site map may be used to adjust one or more of the estimated or predicted values. Such adjusted values can then be used to control the operation of a working implement.

Alternatively or additionally, according to one aspect of the present disclosure, the direct measurements from any of the instruments, particularly the multisensor instrument 300 can be compared to prior measurements for the same or similar worksites, and used to predict one or more operational parameters such as tilling gang angle, tilling depth, planting depth, pesticide application rate, fertilizer rate, seed application rate, or row closure force based on a comparison to the corpus of prior data, such as previously measured soil moisture contents or soil texture values, as correlated to known plant-available water levels or soil water tension values.

According to one aspect of the present disclosure, when data from one of the instruments, including the multisensor instrument 300, the vehicle sensor instrument 402, or the implement sensor instrument 404 is collected across multiple points associated with a specified area, such as a worksite, this data may also be used to prepare a parameter map. For example, data collected by the sensors of the multisensor instrument 300 as previously discussed may be used to calculate a target seed planting depth at a plurality of work locations at a worksite by associating a target seed planting depth value with a corresponding location value. The associated planting depth values and location values can then be compiled to form a planting depth map and corresponding control signals associated with the worksite. It will be further appreciated that, while a method for assembling a planting depth map has been discussed here, substantially similar steps can be followed to prepare maps associating other operational parameters, such as fertilizer rate, pesticide application rate, seeding rate, tilling depth, tiller gang angle, and closing wheel force with locations at the worksite.

According to one aspect of the present disclosure, any directly measured or subsequently calculated value can also be added to an existing corpus of data. The directly measured or subsequently calculated data can be uploaded alone, or with associated location values (for example, it may be uploaded as a map of the relevant data set to a worksite). In some examples, the corpus of data may be associated with the same worksite from which it was gathered. In some examples, the corpus of data may be associated with another worksite, or with a collection of worksites. Advantageously, this may further improve the ability of a system such as the instrumented system 400, to predict (for example, by predictive modeling) one or more operationally significant variable, such as target planting depth, fertilizer rate, pesticide application rate, seeding rate, tilling depth, tiller gang angle, or closing wheel force in a future operation.

According to one aspect of the present disclosure, the operationally significant variables may further be utilized to plan a planting operation. For example, the controller 500 can utilize the available data to predict emergence behavior (for example, emergence time) of a seedling based on a function of any one or more of the measured soil properties, the calculated soil properties, or the calculated and/or predicted operationally significant variables at a plurality of locations at a worksite. Thereafter, one or more operational commands can be executed at each of the plurality of locations at a worksite such that the seedling emerges at all locations at the worksite at the same time, based at least in part on one or more predicted values, such as predicted emergence behavior or predicted plant-available water level. In such an example, for instance, it may be possible to vary planting depth according to the measured soil properties, the calculated soil properties, or the calculated and/or predicted operationally significant variables at the relevant worksite locations, such that the seedling at each location emerges at the same time.

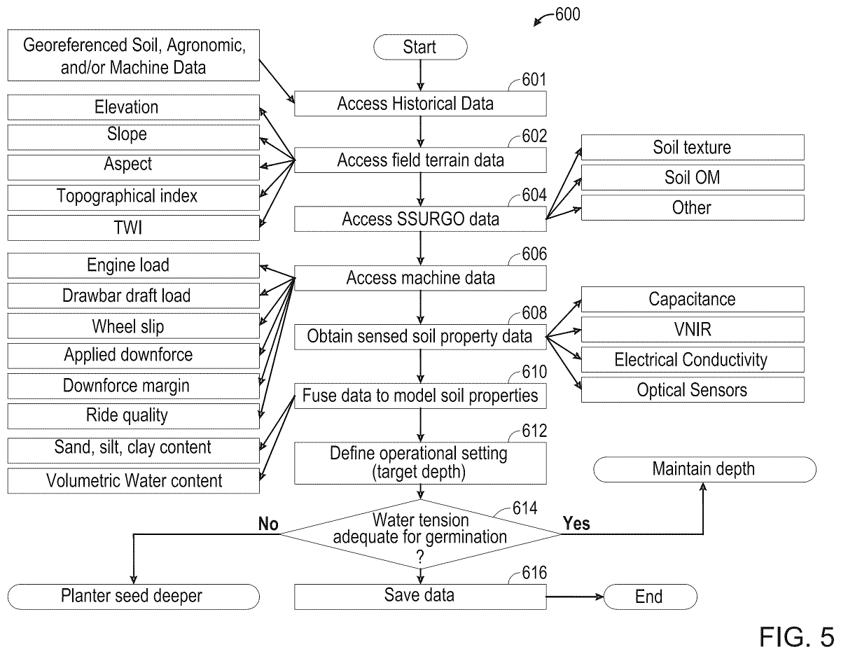

An illustrative example of the methods of implement control disclosed herein is presented in FIG. 5, which shows an instrument controlled operation 600, using an instrumented system such as the instrumented system 400 disclosed herein. While specific reference will be made herein to an instrumented seed applicator, it will be appreciated that similar methods may be applied to an instrument controlled operation of different equipment, such as fertilizing equipment, product application, or tilling equipment.

As shown in FIG. 5, at the start of the instrument controlled planting method 600, the instrumented system can access historical data (process block 601) associated with a work site in which operations will be performed. Examples of historical data relevant to the operations may include various types and combinations of georeferenced soil data, agronomic data, machine operation data, and/or the like as may be appreciated by one of skill in the art. The historical data in question may relate to data captured, derived, or otherwise associated with historical operations by work machines associated with a system and method as disclosed herein, but may further include data related from third party sources. The instrumented system may further access a corpus of field terrain data (process block 602), for example, through an internet connection. The field terrain data can include one or more relevant site characteristics, such as elevation, slope, aspect, topographic wetness index, and the like. This historical data and/or field terrain data can be made available to the controller 500 of the instrumented system 400 and used in the calculation of one or more operational control parameters as discussed further herein.

With continued reference to FIG. 5, the instrument controlled operation 600 can also include a step of accessing publicly available digital soil geographic data (process block 604), for example through an internet connection to the instrumented system 400. The publicly available soil geographic data can include specific relevant soil characteristics, such as soil texture, soil organic matter content, or any other relevant data included in a database accessible to a system as disclosed herein, such as for example a SSURGO database. This data can be made available to the controller 500 of the instrumented system 400 and used in the calculation, estimation, or prediction of one or more soil properties and/or operational control parameters as discussed further herein.

Additionally, the instrumented system 400 can collect data from one or more of the vehicle sensor instruments or implement sensor instruments, such as the vehicle sensor instrument 402 or the implement sensor instrument 404 (process block 606). As previously introduced, the vehicle sensor instrument 402 can provide data on, for example, engine load, draft load, wheel slip, applied machine downforce, downforce margin, and data about ride quality, or any other data relevant to the operation of a work machine. This data can be made available to the controller 500 of the instrumented system 400 and used in the calculation of one or more operational control parameters as discussed further herein.

The instrumented system instrumented system 400 can also collect data from a soil sensor, such as the multisensor instrument 300 disclosed herein (process block 608). For example, the instrumented system 400 can collect data on capacitance, VNIR absorption, temperature, and/or electrical conductivity. In some examples, the 400 can also collect data from optical sensors (for example, in-furrow cameras). This data can be made available to the controller 500 of the instrumented system 400 and used in the calculation of one or more operational control parameters as discussed further herein.

It will be appreciated that, in some cases, one or more of the data access steps indicated in process blocks 601 through 606 may be omitted, depending on the availability of an internet connection, specific sensor packages, and/or the calculations to be performed. Therefore, the instrumented system 400 can access the field terrain data, the SSURGO data, the machine data, and the soil sensor data, or any appropriate combination thereof.

It will be appreciated that, according to some aspects of the present disclosure, this data may also be presented to a vehicle operator, for example, on the user interface 410 of the instrumented system 400. In such examples, the vehicle operator may optionally make one or more user inputs which may be combined with some or all of the data collected.

The data accessed by the instrumented system 400 may thereafter be “fused”, that is, made available to the controller 500 for performing modeling calculations, such as determining calculated soil properties, or calculating or predicting one or more operational control parameters (process block 610). This modeling may produce any one or more of the calculated soil properties or operational control parameters previously identified, including but not limited to soil texture, soil moisture content, modeled or calculated plant-available water level, and modeled or calculated soil water tension value.

Based on the modeling and/or calculation of process block 610, the controller 500 of the instrumented system 400 can thereafter identify one or more operational settings for any one of the instruments in communication with the instrumented system 400 and controlled by the controller 500 (process block 612). Thus, any appropriate combination of the measured or calculated soil characteristics and the predicted or modeled operational control parameters can be used to determine one or more of the target planting depth, fertilizer rate, seeding rate, tilling depth, tiller gang angle, or closing wheel force for a planting operation, and a work tool can be operated based on the operational control parameter. For example, as illustrated in FIG. 5, if the instrument controlled operation 600 is controlling the planting depth of a planting operation, process block 612 can include determining a target seed planting depth, and a seed can be inserted into the soil at the target depth.

According to one aspect of the present disclosure, the instrument controlled operation 600 can include one or more additional steps after the operation of the work tool. For example, in the instrument controlled operation 600 illustrated in FIG. 5, water tension can be measured after the seed is introduced to the soil at a first depth and compared against a known or calculated water tension value required for seed germination. This can be used to determine whether one or more additional steps are required (decision block 614). For instance, in the illustrated example, if the measured or calculated water tension is sufficient for germination, then the seed may be left at the current depth. However, if the measured or calculated water tension is insufficient for germination (that is, less than the target value for soil water tension), then the seeding implement can be advanced further into the soil to a second depth that is deeper than the first depth. After advancing the seed to the second depth, additional measurements can be taken, and water tension values can be recalculated, and further adjustments to seed depth can be made as needed, for example, until the measured plant-available water level matches or exceeds the target value of plant-available water level or modeled plant-available water level.

It will be appreciated that, while the illustrated example includes one or more additional steps that may be appropriate to a seeding operation, a similar logic may be used for any operational parameter used to control the operation of a work tool in communication with the controller 500 of the instrumented system 400, allowing for any executed operation to be adjusted or corrected based on further collected and/or calculated data. It will also be appreciated that in some examples, this additional or corrective step may be omitted.

With continued reference to FIG. 5, after the operation of the work tool has been completed and any required adjustments have been made, any of the measured, calculated, and/or modeled data may thereafter be added to a corpus of data (process block 616). This corpus of data can either previously accessed (for example, the field terrain data and/or the SSURGO data) or may be a new set of data. Thus, for example, data on the soil moisture content value or the soil texture value at one or more locations at a worksite can be added to the corpus of previously measured soil moisture content values or soil texture values.

According to one aspect of the present disclosure, the instrument controlled operation 600 can be executed individually for each relevant position in a worksite. That is data may be retrieved and/or collected, calculations and/or predictions may be made based on this data, and any measured and/or calculated data can be saved to a corpus of data at each relevant location at a worksite or each relevant step of a planned operation. According to another aspect of the present disclosure, data can be retrieved for multiple locations at a worksite and/or multiple iterations of a planned operation in the steps indicated by process blocks 602 through 608, fused in the step indicated by process block 610, and used to calculate operational parameters for the multiple locations and/or iterations as indicated in process block 612, and saved after the multiple iterations of the planned operation have been conducted, as indicated in process block 614.

Thus, although there have been described particular embodiments of the present invention of a new and useful invention, is not intended that such references be construed as limitations upon the scope of this invention except as set forth in the following claims.

What is claimed is:

1. A method for controlling one or more operations of a work machine in a work site, comprising:
generating a first sensor signal and a second sensor signal representative of a first soil characteristic at a work location and a second soil characteristic at the work location, respectively;
calculating a soil moisture content from the first soil characteristic and the second soil characteristic, and calculating a soil texture value from the first soil characteristic and the second soil characteristic;
estimating one or more further soil characteristics based at least on the soil moisture content and the soil texture value;
generating respective values for one or more operational settings based on at least one of the first soil characteristic, the second soil characteristic, and the one or more further soil characteristics; and
controlling at least one operation of the work machine based on the generated one or more operational settings relative to current actual values for the one or more operational settings.

2. The method of claim 1, wherein the second soil characteristic is electrical conductivity.

3. The method of claim 2, further comprising determining a third soil characteristic based on measurements from a third sensor, and estimating the one or more further soil characteristics further based at least on the third soil characteristic.

4. The method of claim 3, wherein the third soil characteristic is a measured absorption value for the absorption of light with wavelengths ranging from 200 nm to 4000 nm, and wherein the calculation of at least one of the soil moisture content and the soil texture value is further based on the measured absorption value.

5. The method of claim 2, further comprising obtaining soil and/or terrain geographic data and/or previously collected proximally-sensed soil sensor information associated with the work site, and estimating the one or more further soil characteristics further based on the obtained soil and/or terrain geographic data and/or previously collected proximally-sensed soil sensor information for respective locations in the work site.

6. The method of claim 2, wherein the first soil characteristic is a dielectric constant, and wherein the measuring of the dielectric constant and the measuring of electrical conductivity is conducted at a plurality of locations, and a soil moisture content and a soil texture value is calculated for each location of the plurality of locations.

7. The method of claim 1, wherein each of the steps are performed by the work machine in association with a current operation.

8. The method of claim 1, further comprising preparing a target planting depth map for a work site including a plurality of locations during a first process associated with the work site, based on a plurality of modeled seed planting depths corresponding to the plurality of locations, and controlling the at least one operation of the work machine during a second process associated with the work site and based on the target planting depth map.

9. The method of claim 8, further comprising transmitting a control signal at each location of the plurality of locations to control the operation of a seeding implement to plant a seed at the modeled seed planting depth corresponding to that location.

10. The method of claim 1, further comprising measuring, with one or more additional sensors, one or more additional soil characteristics and generating one or more corresponding additional signals representative of the one or more additional soil characteristics.

11. The method of claim 10, wherein the step of calculating the soil moisture content from the first soil characteristic and the second soil characteristic, and calculating the soil texture value from the first soil characteristic and the second soil characteristic further includes modifying at least one of the soil moisture content and the soil texture value based on the one or more additional signals.

12. The method of claim 1, wherein the one or more further soil characteristics include at least one of plant-available water level or soil water tension value.

13. The method of claim 12, wherein predicting the plant-available water level or the soil water tension value further comprises comparing the soil moisture content or the soil texture value against a corpus of previously measured soil moisture contents or soil texture values correlated to known plant-available water levels or soil water tension values.

14. The method of claim 13, further comprising adding the soil moisture content or the soil texture value to a corpus of previously measured soil moisture contents or soil texture values and correlating the soil moisture content or the soil texture value to a modeled plant-available water level or soil water tension value.

15. The method of claim 12, further comprising modeling a predicted emergence behavior for a seed planted in a soil having the predicted plant-available water level and soil water tension value.

16. The method of claim 12, wherein the work machine comprises a fertilizer applicator, and the method further comprises:
calculating a starter fertilizer rate based on at least one of the plant-available water level or the soil water tension value and applying a fertilizer to the soil in an amount based on the starter fertilizer rate.

17. The method of claim 12, wherein the work machine comprises a product applicator, and the method further comprises:
calculating a pesticide rate based on at least one of the soil texture value and a calculated organic matter content value, and applying a pesticide treatment in an amount based on the calculated pesticide rate.

18. The method of claim 1, wherein the work machine comprises a tiller, and the operational setting includes a depth or an angle at which a tiller engages the soil based at least in part on the soil moisture content, the soil texture value, or the one or more further soil characteristics.

19. A device configured to be attached to a work machine, the device comprising:
a first sensor configured to generate output signals representative of a dielectric constant of soil;
a second sensor configured to generate output signals representative of soil conductivity; and
a controller operably connected to the first and second sensors, and a seed applicator or a tiller, and configured to direct performance of the method of claim 1, wherein the dielectric constant is the first soil characteristic and the soil conductivity is the second soil characteristic.

20. A system for controlling one or more operations of a work machine in a work site, the system comprising:
a first sensor configured to generate output signals representative of a dielectric constant of soil;
a second sensor configured to generate output signals representative of soil conductivity; and
one or more processors operably connected to the first and second sensors, and configured to direct performance of the method of claim 1, wherein the dielectric constant is the first soil characteristic and the soil conductivity is the second soil characteristic.
